(19) United States
(12) Patent Application Publication
(10) Pub. No.: US 2009/0048577 A1
(43) Pub. Date: Feb. 19, 2009

(54) Multi-Port Catheter System with Medium Control and Measurement Systems for Therapy and Diagnosis Delivery

(75) Inventors: George T. Gillies, Charlottesville, VA (US); Richard E. Latchaw, Sacramento, CA (US)

Correspondence Address: UNIVERSITY OF VIRGINIA PATENT FOUNDATION, 250 WEST MAIN STREET, SUITE 300, CHARLOTTESVILLE, VA, 22902, US

(73) Assignees: UNIVERSITY OF VIRGINIA PATENT FOUNDATION, Charlottesville, VA (US); THE REGENTS OF THE UNIVERSITY OF CALIFORNIA, Davis, CA (US)

(21) Appl. No.: 12/160,378

(22) PCT Filed: Jan. 9, 2007
(86) PCT No.: PCT/US07/00353

§ 371 Date: Aug. 1, 2008

Related U.S. Application Data
(60) Provisional application No. 60/757,266, filed on Jan. 9, 2006.
(60) Provisional application No. 60/757,271, filed on Jan. 9, 2006.

(51) Int. Cl. A61M 25/09; A61B 6/00; A61M 25/00; A61B 5/05

(52) U.S. Cl. 604/510; 600/435; 604/523; 600/420; 600/434; 604/508

(57) ABSTRACT

A series of embodiments of multi-lumen, multisegmented (or variable diameter) catheters and associated multi-channel flow control and measurement systems and methods for simultaneous delivery of a medium to a plurality of locations is described. The need for precise, stable reliable, and repeatable flow control in therapy delivery catheters is crucial to the efficacious treatment of the clinical manifestations of peripheral vascular disease (PVD) and other such maladies. Such treatments may involve the placement of multi-lumen catheters into peripheral arterial trees, with the subsequent need to govern the flow dynamics within each individual lumen of the multi-lumen device in such a way that an optimum distribution of the agent is achieved intra-arterially. Combinations of pumps, flow monitors, pressure monitors, feedback loops and related hardware and software collectively capable of achieving this goal are described. In other embodiments, this device and method could be used for infusions into tissues and solid organs, and microcoil systems can be added to the various components of the catheter systems to improve the imaging quality during MR-guided procedures.

### RELATED APPLICATIONS

The present application claims priority from U.S. Provisional Application Ser. No. 60/757,271, filed Jan. 9, 2006, entitled “Multi-Segment/Multi-Port Catheter for Use in the Peripheral Vasculature,” and Ser. No. 60/757,266, filed Jan. 9, 2006, entitled “Multi-Channel Flow Control and Measurement Systems for Therapy Delivery Catheters,” the disclosures of which are hereby incorporated by reference herein in their entirety.

### BACKGROUND OF THE INVENTION

Peripheral vascular disease (“PVD”), like coronary artery disease (“CAD”), is the progressive narrowing of the arterial tree by the atherosclerotic process which results in diminished blood flow to vital organs and extremities beyond the site of narrowing or occlusion. Diabetes mellitus (“DM”) is a major factor in this disease process, and as the prevalence of DM increases, so does that of PVD and CAD. PVD affects an estimated 27 million people in Europe and North America, and it produces significant morbidity and mortality. An estimated 10.5 million of those affected are symptomatic while 16.5 million are asymptomatic. Despite the prevalence of PVD, it is estimated that only 25 percent of symptomatic patients are currently treated for the disease.

PVD typically affects multiple segments of a given artery. Short segments of severe narrowing are typically treated with catheter-based techniques such as angioplasty and stenting. When there is severe narrowing over a long segment or involving multiple arteries to a limb, surgical revascularization is the treatment of choice. When this is insufficient, particularly in the diabetic population, limb amputation, of which an estimated 60,000 are performed annually in the United States, may become necessary. Severe narrowing or poor flow commonly result in the formation of intra-arterial thrombus (clot), which, if not immediately corrected, will lead to the death of tissue and amputation.

Systemic administration of therapeutic agents allows for widespread distribution of these agents throughout the body. The function of the therapeutic agents depends upon the uptake of the medication by the targeted organ and upon the agent's pharmacokinetics which determine its concentration: In addition, with systemic delivery, non-targeted organs may be adversely affected by the medication, leading to potentially serious side effects. Consequently, the efficacy of the therapeutic agents at the target location can be limited by both its concentration at the location of interest and its toxicity in other non-targeted organs.

An emerging market for the treatment of PVD is site-specific stem cell therapy for the treatment of ischemic limbs. This cellular therapy has demonstrated efficacy in the formation of new blood vessels in ischemic limbs of patients with PVD in a recently published randomized controlled clinical study. The increasing population of patients with DM and PVD potentially makes this a very large market.

The conventional devices presently used in this field do not allow for sufficiently selective flow control and measurement of the concentrations of diagnostic agents and anti-thrombolytic agents in specific regions of peripheral vasculature that are diseased or obstructed. An example of this type of limitation in the prior art relevant to this field is U.S. Pat. No. 6,800,075 by Mische and Beck (of which is hereby incorporated by reference herein), which discloses a multi-lumen catheter for delivery of therapeutic agents and removal of debris from vascular structures, but does not teach any methods of flow control for multi-lesion treatments using the same device. A further limitation of the prior art is that the flow-driving devices sometimes incorporate complex valving methods which require an elevation differential between the distal end of the fluid line (catheter) and the fluid flow control device, as in U.S. Pat. No. 6,808,369 by Gray and Bryant (of which is hereby incorporated by reference herein). Another limitation of the prior art in the field of multi-lumen catheters has to do with the inherent inability of existing designs to provide the clinical user with feedback signals to continuously control the relative flow rates through two or more ports along the length of the device, which limitation arises for instance in the method and device described in U.S. Pat. No. 6,808,510 by Difiore (of which is hereby incorporated by reference herein) and in U.S. Pat. No. 6,824,532 by Gillis and Theeuwes (of which is hereby incorporated by reference herein). Still another limitation of the existing art in the field of multi-lumen devices centers on the inability of the system- to delivery only one agent at a time, as would be the case for the device and method of U.S. Pat. No. 6,819,951 by Patel et al. (of which is hereby incorporated by reference herein), as well as the device and method of U.S. Pat. No. 6,827,710 by Mooney et al (of which is hereby incorporated by reference herein). Another limitation of the existing art lies in the inability of recently described flow control devices to provide the flow patterns that would characteristically be needed to achieve appropriate concentrations of the therapeutic agent in the vicinity of complex vascular lesions. Examples of devices and methods limited in this regard are the nonlaminar flow system described in U.S. Pat. No. 6,830,563 by Singer (of which is hereby incorporated by reference herein) and the flow reversal device and method disclosed in U.S. Pat. No. 6,830,579 by Barbut (of which is hereby incorporated by reference herein). A particularly important limitation of the existing art has to do with the inability of flow management systems for catheters to distinguish between variations in delivery caused by pressure excursions versus those caused by flow disruptions. Examples of the methods and devices so constrained in their performance are those described in U.S. Pat. No. 6,834,242 by Wolff and Rondelet (of which is hereby incorporated by reference herein), and in U.S. Pat. No. 6,834,842 by Houde (of which is hereby incorporated by reference herein). Moreover, none of the devices in the existing art are optimized in their design to include microcoil systems for imaging enhancement, thus adding a further limitation to their performance characteristics.

There are several existing patents which claim multi-lumen catheters which use an outer and inner tube. One such patent is U.S. Pat. No. 6,755,813 by Ouriel et al. (of which is hereby incorporated by reference herein), which claims a tube-within-a-tube catheter system. Yet devices such as this one do not allow for simultaneous delivery of a medium to a plurality of locations, nor do they allow for diagnostic contrast agents to be delivered at the same time as a lysing medium.

### SUMMARY OF THE INVENTION

While conventional endovascular catheter placement for the delivery of a thrombolytic agent to dissolve the clot may be efficacious to some extent, they commonly require days of drug infusion, intensive care monitoring, and frequent trips to the radiology department to reposition the catheter.

Regarding an aspect of various embodiments of the present invention, the endovascular regimen may include the placement of a catheter throughout the entire length of the thrombus without the need for catheter repositioning. Through this catheter, a thrombolytic agent could be delivered to the affected arterial regions (or other applicable regions) simultaneously or intermittently, potentially minimizing the duration of the therapy.

The clinical benefits of flow-controlled, site-specific catheter-based delivery systems/methods of an aspect of various embodiments of the present invention for the administration of therapeutics may include increased safety, increased efficacy, reduced toxicities, more reliable therapeutic drug levels, and decreased and simplified dosing requirements. Safety, efficacy, and toxicity are all nominally independent variables, but are functionally related parameters in the pharmacokinetics of each therapeutic agent. Site-specific drug delivery into the target tissue ensures that the majority of the drug goes to the site it is intended to act upon with minimal or small effect upon non-targeted tissue, thereby decreasing toxicity. This allows higher concentrations of the therapeutic agent to be administered to the targeted site, thereby increasing efficacy. An additional benefit of site-specific delivery of therapeutic agents is that the patient receives a smaller cumulative dose, thereby increasing safety.

An aspect of various embodiments of the present invention provides a site-specific catheter-based drug delivery that allows local administration of therapeutic agents and reliable therapeutic drug levels to be achieved and maintained because systemic clearance is reduced. By obtaining reliable therapeutic drug levels in this manner, dosing requirements are decreased and simplified. As mentioned above, local drug levels can be maintained at higher levels than could be achieved with systemic administration because systemic toxicity is reduced with local delivery.

Precise control of the flows through multi-lumen and single-lumen site-specific drug delivery catheters is required when active biologic agents are being administered to focal locations. As an example, site-specific delivery of thrombolytic therapy to the location of a clot in the vascular tree of an ischemic limb is preferred to systemic delivery. Careful control of the flow of the therapeutic agent is required to achieve clot lysis at the location of the infusion, whereas, systemic delivery of thrombolytic therapy could lead to generalized bleeding at multiple remote locations.

An aspect of an embodiment provides a design for a catheter system and method for the treatment of peripheral arterial thrombotic disease such as PVD, such as in the leg of a subject, as well as other locations of the subject according to anatomical and physiological requirements as will be discussed herein. This device, termed the “PeriCATH”, is an infusion catheter that can reliably deliver therapeutic agents and/or diagnostic agents simultaneously to multiple locations of vascular lesions that extend over long segments of an organ or tubular structure, such as blood vessels.

In an approach, the PeriCATH uses a flow control and measurement system that is designed for use with catheters and related implantable devices employed to treat peripheral arterial thrombotic disease, such as in the leg or other applicable anatomy locations if desired or required. In a general exemplary embodiment, the system includes a control loop synthesized to regulate the line pressures in and flows through the independent channels of a therapy delivery device such as a multi-segmented/multi-lumen catheter used to deliver PVD treatments. The efferent components of the control loop may include a medium control system which may comprise flow actuators such as pumps, in-line pressure and flow sensors, connector tubes, valves, a guidewire to position the catheter at the target area, and the catheter itself. The catheter is composed of an outer tube and inner tube, which may be a guide catheter and a delivery catheter, respectively. The inner tube is adapted to move circumferentially and/or longitudinally relative to the outer tube to allow transference of the medium between the inner tube and the plurality of locations within the body. The outer and inner tubes may have apertures through which a medium may be delivered or withdrawn, or they may be made of a semipermeable membrane. The apertures, if any, may be port holes, conduits, or permeable or semi-permeable structures.

The afferent components and elements of the control loop include the imaging system needed to observe the volume of distribution of the infused agent; the host computer or computational platform needed to acquire and process the imaging and hydrodynamic, the user who may be a doctor or a clinician and who commands and regulate the flow actuators; and the control algorithm employed by the user to optimize the treatment to the needs of the particular patient, who may be a human or animal.

One skilled in the art will recognize that these components and system provide a unique and previously undisclosed means and technique for generating a customized volume of distribution of the infused agent within the region of PVD-related lesions within a peripheral arterial tree of a subject (or as applied to an organ or tubular structure of a subject), who may be a medical patient. The catheter component of the system can be made available for use with or without microcoils wound onto it in preferred locations, for use in enhancing the performance of the imaging system.

An aspect of various embodiments of the present system and method may be used for simultaneous delivery (as well as scheduled or temporarily desired/required) of thrombolytics or other mediums to multiple locations, as well as the delivery of diagnostic contrast agents in tandem with such mediums.

It should be appreciated that PeriCATH is not necessarily limited to the arterial structure. It may also be used in the organ structures or tubular structures. An organ includes, for example, a hollow organ, parenchymal tissue (e.g., brain, kidneys, liver, etc.) and/or stromal tissue Hollow organ structures includes, for example, stomach, esophagus, colon, rectum, and ducts, or the like. A tubular structure may include a blood vessel. A blood vessel may include one or more of the following: vein, venule, artery, arterial, or capillary.

An aspect may provide a series of embodiments of multi-lumen, multisegmented (or variable diameter) catheters and associated multi-channel flow control and measurement systems and methods for simultaneous delivery of a medium to a plurality of locations is described. The need for precise, stable reliable, and repeatable flow control in therapy delivery catheters may be crucial to the efficacious treatment of the clinical manifestations of peripheral vascular disease (PVD) and other such maladies. Such treatments may involve the placement of multi-lumen catheters into peripheral arterial trees, with the subsequent need to govern the flow dynamics within each individual lumen of the multi-lumen device in such a way that an optimum distribution of the agent is achieved intra-arterially. Combinations of pumps, flow monitors, pressure monitors, feedback loops and related hardware and software collectively capable of achieving this goal are implemented and utilized. In other embodiments, this device and method could be used for infusions into tissues and solid organs, and microcoil systems can be added to the various components of the catheter systems to improve the imaging quality during MR-guided procedures.

An aspect of an embodiment of the present invention device and method may provide flow control for multi-lesion treatments using the same device.

An aspect of an embodiment of the present invention device and method may provide the clinical user with feedback signals to continuously control the relative flow rates through two or more ports along the length of the device.

An aspect of an embodiment of the present invention device and method may provide ability of the system to delivery one or more agents at a time (or temporally as desired), as well as one or more agents at one or more locations at a time (or temporally as desired). Accordingly, the method and system may comprise transferring a therapeutic agent and/or a diagnostic agent separately to two or more different pluralities of locations in the body.

An aspect of an embodiment of the present invention device and method may provide flow control devices/modules to provide the flow patterns that would characteristically be needed to achieve appropriate concentrations of the therapeutic agent in the vicinity of complex vascular lesions or other anatomical regions, structures, regions or characteristics.

An aspect of an embodiment of the present invention device and method may provide the ability of present invention's flow management system of the catheters to distinguish between variations in delivery caused by pressure excursions versus those caused by flow disruptions.

An aspect of an embodiment of the present invention provides a catheter system for transferring at least one medium into a plurality of locations within a body of a patient. The system may comprise, but not limited thereto, the following: 1) an outer tube having a distal end and a proximal end, and having a wall extending longitudinally there between; 2) at least one inner tube disposed within the outer tube, wherein at least one inner tube having a distal end and a proximal end, and having a wall extending longitudinally there between; 3) wherein the at least one inner tube being adapted to move longitudinally and/or circumferentially relative to the outer tube to move along the body to allow transference of the medium between the inner tube and the plurality of locations within the body; 4) an imaging system to provide imaging data; and 5) a medium control system to control the medium.

An aspect of an embodiment of the present invention provides a method for transferring at least one medium into a plurality of locations within a body of a patient using a catheter system. The method may comprise, but not limited thereto, the following: 1) inserting an outer tube into the body, wherein the outer tube having a distal end and a proximal end, and having a wall extending longitudinally there between; 2) inserting at least one inner tube disposed within the outer tube, wherein at least one inner tube having a distal end and a proximal end, and having a wall extending longitudinally there between; 3) wherein at least one inner tube being adapted to move longitudinally and/or circumferentially relative to the outer tube to move along the body to allow transference of the medium between the inner tube and the plurality of locations within the body; 4) imaging data using an imaging system; and 5) controlling the medium.

These and other objects, along with advantages and features of the invention disclosed herein, will be made more apparent from the description, drawings and claims that follow.

### BRIEF DESCRIPTION OF THE DRAWINGS

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIGS. 1(A)-(C) are schematic illustrations of exemplary components of the catheter system, demonstrating the inter-relationships between them. The embodiment of the system represented by the diagram of FIG. 1(A) provides an inner and outer tube connected to the medium control system which itself is connected to an imaging system. FIG. 1(B) displays a non-limiting embodiment of the medium control system in more detail. FIG. 1(C) illustrates three independent channels through which therapeutic agents could be delivered (or medium withdrawn).

FIG. 2 is a schematic elevation view of a portion of a catheter having an outer catheter or outer tube which has an empty inner barrel into which an inner catheter or inner tube (not shown) can be inserted, removed, translated and/or rotated therein. Also shown are the apertures which can be unoccluded by rotation and extension of the inner tube (not shown) relative to the outer tube.

FIGS. 3(A)-(B) are schematic elevation views of a portion of a catheter having an outer catheter or tube disposed within an inner catheter tube wherein their respective apertures are misaligned and aligned, respectively. FIG. 3(C) additionally shows that the inner tube can be extended out beyond the distal end of the outer tube.

FIG. 4 is a schematic illustration of exemplary components of the catheter system, demonstrating the inter-relationships between them, and having three independent channels, i.e. non-communicating channels, through which therapeutic agents or diagnostic agents could be delivered (or medium withdrawn).

FIG. 5 is an enlarged partial view of exemplary features of the construction of one embodiment of the drug delivery catheter.

FIG. 6 is a schematic illustration of one scenario for the spread of the infusate following delivery of it through a port hole of the catheter.

FIG. 7 provides one possible version of the general class of algorithms that may be employed to control the overall system via the computer, processor or computational platform.

FIG. 8(A) is a photographic depiction of a section of a peripheral vascular tract in a patient, into which an anti-thrombolytic agent may be delivered. FIG. 8(B) is a photographic depiction of a multi-segmented, multi-port PeriCATH device that has been navigated into the section of peripheral vasculature for the delivery of the agent.

FIG. 9(A) is a schematic depiction of a tapered construction of the PeriCATH device, along with its position within an artery that has thrombal obstructions within it. For example, a multi-lumen or single lumen PeriCATH device has its inner tube that is slid through an outer “sleeve” tube to expose the port holes to a proximal obstruction that must be treated. FIG. 9(B) shows the same device of FIG. 9(A) with an anti-thrombolytic agent being selectively delivered to the proximal obstruction in the artery via selected port holes in the inner tube.

FIG. 10 is a schematic diagram showing a patient, or any subject or object, undergoing an examination and/or intervention in an MRI magnet whereby a catheter device is disposed at any of one or more desired or required locations within the patient.

### DETAILED DESCRIPTION OF THE INVENTION

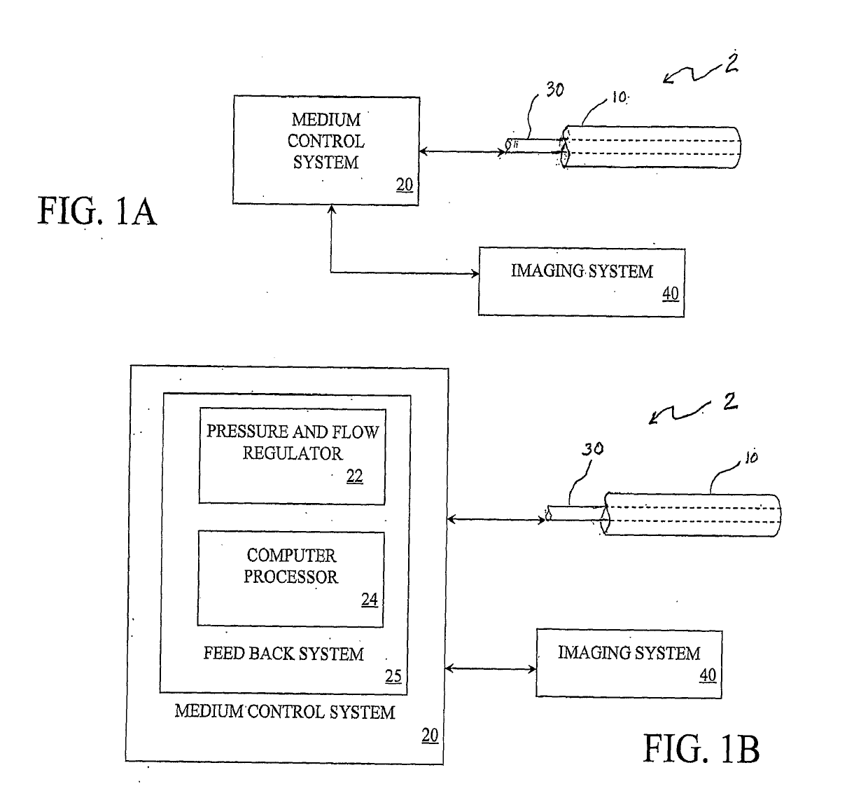

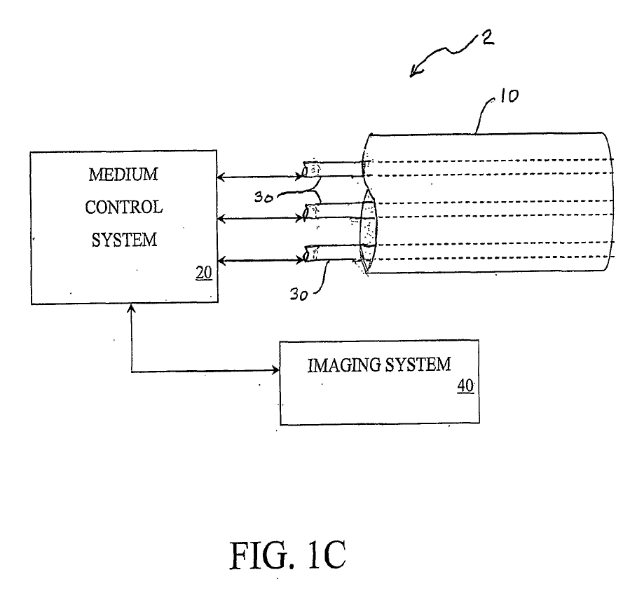

FIG. 1(A) is a schematic illustration of an embodiment of the catheter system 2. The catheter system 2 comprises an outer tube 10, or guide catheter, within which is an inner tube 30, or delivery catheter. This catheter system 2 is used to deliver (or withdraw) a medium through the use of a medium control system 20, which may monitor the process through the attached imaging system 40.

FIG. 1(B) schematically illustrates the medium control system 20 of FIG. 1(A), wherein the medium control system 20 comprises a feed back system 25 that may comprise a pressure and flow regulator 22, controlled by a computer processor 24, for example, which can automate the process of transferring a medium to a plurality of locations in the subject 4 (not shown). The imaging data from the imaging system 40, and the pressure and flow-rate data from each inner catheter 30 may be fed into the host computer or processor 24. An algorithm in the host computer or processor 24 may be used to process the data. The user operating the system can then input commands to the host computer 24 to modify the flow rate and/or pressure in each of the independent channels via control signals from the computer 24 to each of the pumps 26 (not shown), for example. The end result is intended to be the confirmed delivery of therapeutically or diagnostically needed concentration of the medium within the vicinity of each PVD lesion 8 (not shown) or other applicable anatomy location of patient to be treated, with the volume of distribution of the medium visualized by the imaging system 40. Full automation of the entire system via the host computer 24 can enable the system to correct for deviations in the as-imaged volume of distribution by making adjustments to the in-line pressures and flow rates.

FIG. 1 (C) schematically illustrates the medium control system 20 of FIG. 1(A), with the addition of multiple delivery catheters 30 (inner tubes) being disposed in the guide catheter 10 (outer tube). This is a multi-lumen system which can be used to deliver (or withdraw) multiple mediums at once or sequentially (or any temporal relationship as desired or required). These mediums can include therapeutics for treating lesions 8 (not shown), and contrast agents for diagnostics and imaging, and other types of agents, mediums, medications, and fluids. These mediums can be delivered to (or withdrawn from) a plurality of locations simultaneously, such that, for example, one location can be undergoing diagnosis while another is receiving therapeutics for lysing of a lesion.

It should be appreciated that any of the components or modules referred to in FIGS. 1(A)-(C), may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwire or other desirable and available communication means, systems and hardware.

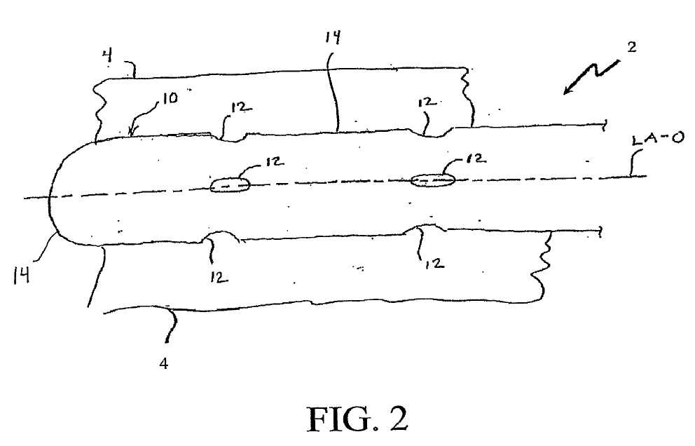

FIG. 2 is a schematic elevation view of a portion of a catheter system 2 having an outer catheter 10 or outer tube which has an empty inner barrel into which inner catheter 30 or an inner tube (not shown) can be inserted, removed, translated and rotated during the course, for instance, of progressive lysis of arterial lesions. It should be appreciated that the outer tube 10 may be moved, translated or rotated. The outer catheter 10 or outer tube has an arrangement of apertures 12 such as a port hole means placed in its wall 14 in an appropriate or desired pattern. In one specific embodiment as shown here, the port holes 12 are in a circumferential arrangement, with one or more sets of them located along the longitudinal axis, LA-O, of the outer catheter 10. The apertures 12 may be arranged and located circumferentially and longitudinally as required.

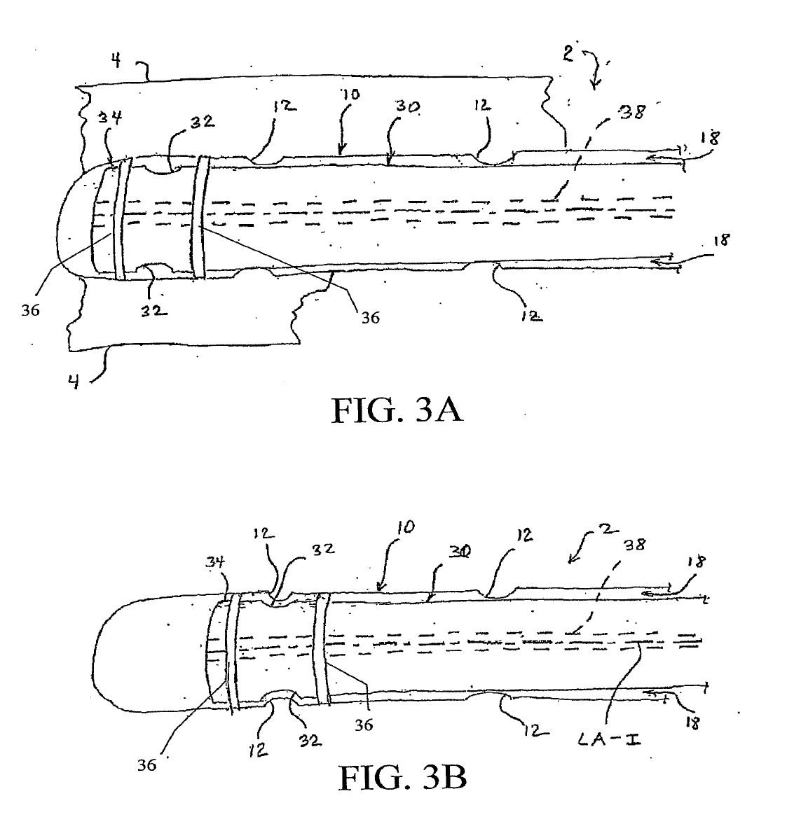

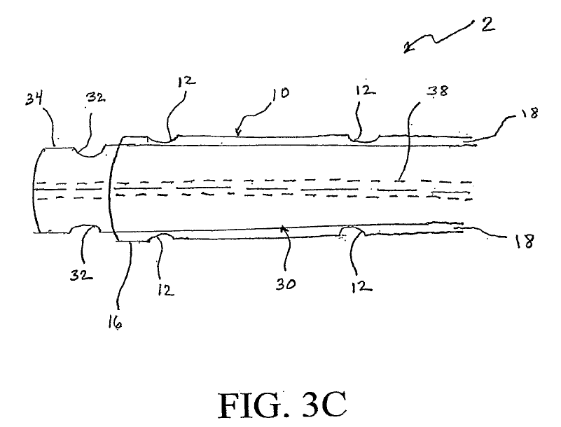

FIGS. 3(A)-3(C) are schematic elevational views showing that the inner catheter 30 or inner tube may be inserted, removed, translated, or rotated within the outer catheter 10, and typically will be substantially coaxially aligned with the outer catheter 10 although that is not a requirement. The space between the outer wall of the inner catheter 10 and the inner wall of the outer catheter 30 is defined as an inter-tube gap 18, which is not necessarily required. The inner catheter 30 has at least one or more apertures, such as port hole means 32, located circumferentially at or proximal to its distal tip/end/region 34. The distal tip/end 34 of inner catheter 30 may have an optional sealing device or system 36 (or other structure to block the transference of a given medium) such as a plurality of circumferential gaskets located on either side of the aperture or port hole means 32, as taught in PCT Application No. PCT/US2005/026738, filed Jul. 28, 2005, and corresponding U.S. application Ser. No. 11/191,676, filed Jul. 28, 2005, of which are hereby incorporated by reference herein in their entirety. This sealing device is an optional element.

In one alternative specific embodiment, the sealing device or system might be similar to gaskets, o-ring seals or another type of annular means that projects above the surface of the inner catheter, coplanar with the surface of the catheter, below the surface of the outer catheter or otherwise positioned within the inter-tube gap. Other examples of the sealing device or function may be provided by, but not limited thereto, the following: sleeve, grommet, bushing, annular rivet, snap closure, slip, pressure seal, elastic seal, pneumatic tension seal, collar, engaged seal, engaged joint, tab, offset, protuberance, shelf, ledge, extension, lip, bulge, collet, flange, thimble, ring, knob, or a friction-fit communication between the inner tube and outer tube, elastic conformance, threaded-fit, bayoneted-fit, or other types of locking mechanisms or circumferential sealing devices or systems. Among the functions that may be provided by this sealing system is the reduced ability of materials to flow into and out of a hole in the outer catheter (tube), where such flow is not intended. For example, if there were a gap between the inner catheter (tube) and the outer catheter (tube), and holes in the inner catheter and outer catheter were aligned to allow the diffusion or spread of materials released from the inner catheter through the hole in the outer catheter, materials in the environment to which said diffusion or spread is to be effected could reflux into a space between the inner and outer catheters and materials trapped or located in that space between the inner and outer catheters could migrate or otherwise transfer into that environment or be released at another unintended time and location. One function of the seal system or device is to reduce any such unintended capture and/or release of materials, including materials present in space between the inner and outer catheter. The seal may also operate to reduce mass transfer of materials back into the openings in the inner and outer catheters, preventing other spurious concentration changes in materials to be delivered and/or reduce dilution of materials to be delivered and/or prevent spurious transport of materials from one environment to another environment by being picked up through transfer into space carried in or around the inner and/or outer catheter.

For various embodiments the inner catheter 30 will generally have multiple inner chambers, channels or lumens each constituting a separate lumen of the device that communicates with an inlet on the proximal end/tip/region 35 (not shown) of the inner catheter 30. One example of such an inner chamber is shown as an intra-inner tube lumen 38. Referring to FIG. 3(A), the inner catheter 30 and outer catheter are shown positioned or located such that the inner catheter aperture(s) 32 and outer catheter aperture(s) 12 are not aligned in a manner so as to prevent any transference of medium there through. For example, the medium could not be transferred or exchanged between the subject and the inner catheter/tube 30 via the pathway of the inner catheter apertures 32 and outer catheter apertures 12 combination. Alternatively, Referring to FIG. 3(B), the inner catheter 30 and outer catheter are shown positioned or located such that the inner catheter aperture(s) 32 and outer catheter aperture(s) 12 are at least partially aligned or congruent with one another such that a medium can be transferred between the inner tube 30 and the subject 4 via the pathway of the inner catheter aperture(s) 32 and outer catheter aperture(s) 12 combination. Some examples of medium that may be transferred from the inner tube to the subject may include, but not limited thereto, the following: therapeutic and diagnostic agents, for example, thrombolytic agents, chemotherapies, cell slurries, gene therapy vectors, growth factors, contrast agents, angiogenesis factors, radionuclide slurries, anti-infection agents, anti-tumor compounds, receptor-bound agents, cleansing or lavaging agents, and/or other types of drugs, therapeutic and/or diagnostic agents, and other such substances.

Similarly, some examples of medium that may be transferred (i.e., withdrawn) from the subject to the inner tube may include, but not limited thereto, the following: edematous fluids, blood, cerebrospinal fluid, interstitial fluid, infected materials, neoplastic fluids and tissues, thrombolysis byproducts including clot fragments and the like, metabolic byproducts, excess drugs and agents, and other such substances.

It should be appreciated that the inner catheter tube 30 and outer catheter 10 tube may be comprised of a variety structures including, but not limited thereto, the following: various types of conduits, channels, passages, pipes, tunnels, and/or bounded tubular surfaces or the like. Moreover, the tubes may have a variety of cross-sectional shapes including, but not limited to the following geometric shapes: circular, oval, multi-faceted, square, rectangular, hexagonal, octagons, parallelogram hexagonal, triangular, ellipsoidal, pentagonal, octagonal, or combinations thereof or other desired shapes, including variable diameter or cross-section geometries and irregular geometries.

Further, it should be appreciated that any of the apertures discussed herein may have a variety of shapes such as, but not limited thereto, the following circular, oval, multi-faceted, square, rectangular, hexagonal, octagons, parallelogram hexagonal, triangular, ellipsoidal, pentagonal, octagonal, or combinations thereof or other desired shapes.

Similarly, the apertures discussed herein may be of a variety structures such as, but not limited thereto, the following: recess, port, duct, trough, bore, inlet, hole, perforation, channel, passage, slot, orifice, semipermeable membrane, or the like.

Moreover, it should be appreciated that the various components of the inner and outer catheter may be a variety of commercially available materials used for all types of catheter systems. Some examples of materials used for the inner and outer catheters may include, but not limited thereto, the following: polymers, rubbers, plastic, composites, metals, ceramics, hydrogels, dialysis membranes and other membranous materials, MR-compatible alloys and materials, and other organic and inorganic compounds and substances and the like. It should be appreciated that the various components of the catheter system 2, including but not limited thereto, the outer tube 10, sealing device 36 and inner tube 30 may be flexible or rigid and combination thereof as required or desired for intended use. Similarly, the catheter system 2, including but not limited thereto, the outer tube 10, sealing device 36 and inner tube 30 may provide volume contoured delivery/withdrawal (i.e., transfer) of a medium by adjusting its geometry and flexibility/rigidity according to the target location or anatomy (or region, including structure and morphology of any lesion) being treated.

Referring to FIG. 3(C), the inner catheter 30 is extended beyond the distal end 16 of outer tube 10 as desired or required, thus allowing mediums to be transferred to locations directly from the distal end 34 of the inner catheter or the inner tube aperture 32.

Still referring to FIGS. 3(A)-3(C), an inner tube end port may be provided (not shown). It should be appreciated that the inner tube apertures, outer tube apertures, and end ports may be arranged circumferentially and longitudinally, as well as omitted, as desired or required.

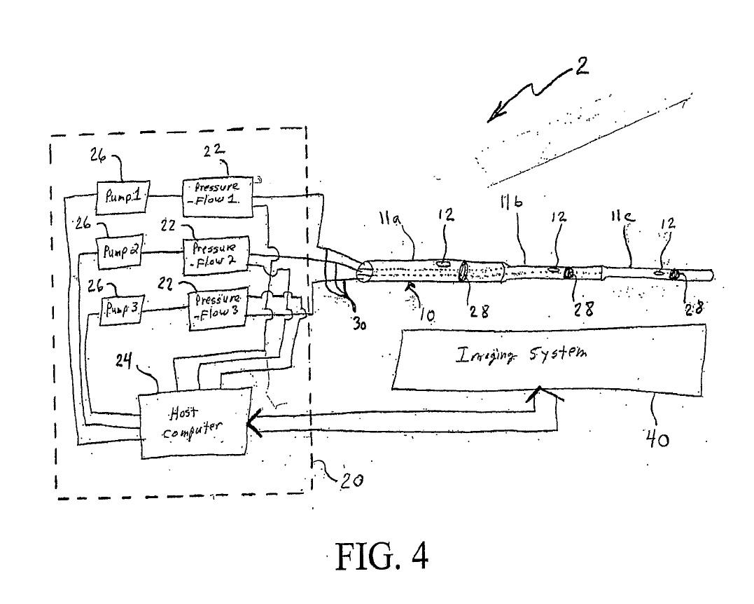

FIG. 4 is a schematic illustration of the catheter system 2 with a multi-segmented catheter outer tube 10 having multiple segments 11a, 11b and 11c (number of segments as desired or required) each having respective maximum diameters. For example, but not limited thereto, the largest segment 11a may be used within the superficial femoral artery, the middle segment 11b may be used for the popliteal, and the smallest segment 11c may be used for the “run-off” vessels in the lower leg. These three segments may gradually taper into each other. In an embodiment, their approximate diameters are 5.0 F, 3.5 F, and 2.0 French respectively. It should be appreciated that any catheter diameter size or length may be utilized as required or desired according to medical procedure/treatment or anatomical location or physical requirement.

Still referring to FIG. 4, for illustrative purposes, one aperture 12 is shown on each of those three segments 11a, 11b and 11c, of the guide catheter outer tube 10, and the windings of a microcoil 28 for imaging enhancement are shown next to each aperture 12. The catheter's location in the limb of the patient 4 (not shown) and the distribution of the medium pumped through each aperture 12 are observed by an appropriate imaging system 40. The catheter 2 is divided axially into three non-communicating internal lumens 30, one capable of delivering flow into each of the three segments 11a, 11b and 11c, of the catheter 2. The medium control system 20 may comprise a separate pump 26 that drives the flow into each of the three lumens 30. The pressure in each of those lines and the flow rate through it is monitored by dedicated instrumentation modules such as pressure and/or flow regulators 22. The imaging data from the imaging system 40, and the pressure and flow-rate data from each infusion line 30 are fed into the host computer or processor 24. An algorithm or processor in the host computer 24 may be used to process the data. The clinician operating the system 2 may then input commands to the medium control system 20 or host computer 24 to modify the flow rate and/or pressure in each of the independent channels 30 via control signals from the computer 24 to each of the pumps 26. In an embodiment, the end result provides the confirmed delivery of therapeutic concentration of the infusate within the vicinity of each PVD lesion 8 (not shown) or other anatomy location of patient to be treated, with the volume of distribution of the infusate visualized by the imaging system 40. Full automation of the entire system via the host computer 24 or processor enables the system to correct for deviations in the as-imaged volume of distribution by making adjustments to the in-line pressures and flow rates.

It should be appreciated that the imaging system may be replaced, augmented and/or automated with a computer processor (or applicable computer hardware) or software that may obtain and handle the data that an imaging system would process/handle and viewed or interpreted by clinician/user/operator, etc.

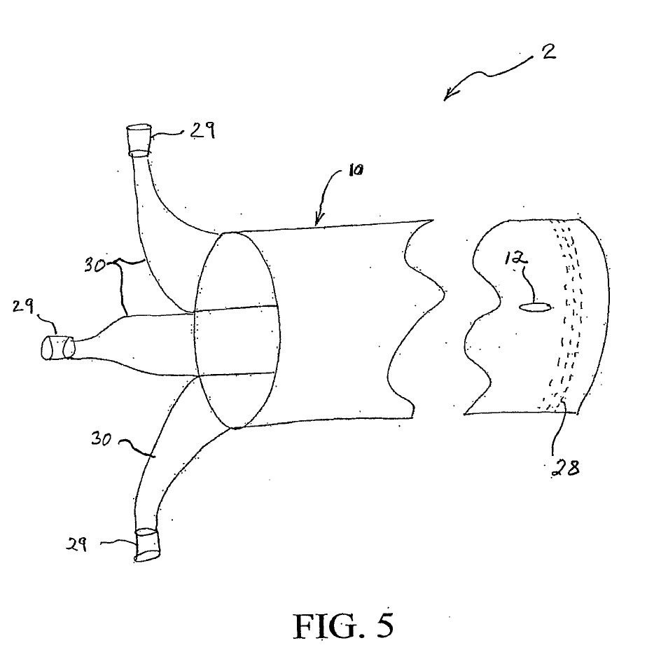

Turning to FIG. 5, FIG. 5 is an enlarged partial schematic view of the catheter outer tube 10 revealing that three separate tubing sections 29 that are each connected each of the three independent internal lumens 30 of the outer catheter 10. Each of the tubes of the internal lumens 30 may have Luer fittings on its proximal end to facilitate interconnection of the inner catheter tube 30 to the rest of the therapy delivery system. Also shown in FIG. 5 is an axial or longitudinal segment of the catheter outer tube 10 showing a port hole 12 in proximity to the windings of the microcoil 28. The port holes 12 or the like and microcoils 28 or the like may be located or positioned relative to one another as desired or required for operation of the catheter system.

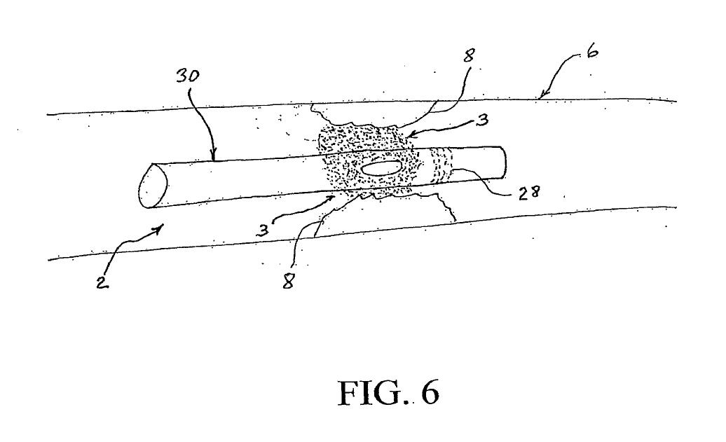

Referring to FIG. 6, FIG. 6 is a schematic illustration of one scenario for the spread of the infusate following delivery of it through a port hole 12 of the inner tube catheter 30. The infusion may occur through at least one the port holes 12 of at least one segment of the catheter 2. As illustrated, the inner tube catheter 30 is inside the lumen of a blood vessel 6 and the port hole 12 is situated within the constricted region of a PVD-related lesion 8 (or any designated location or region of applicable anatomy of the patient under subject therapy or diagnosis) that partially occludes the vessel 6 (or subject location or region). The agent is seen to form a cloud 3 around the port hole 12 in proximity of the lesion 8, thus enabling the dissolution of the lesion 8. It should be appreciated that the infusion may occur through one or more port holes at one or more segments using one or more inner catheter tubes and which the infusion/delivery may occur simultaneously at one or more locations or with any temporal relationship, such as sequentially or intermittently, as designated by the user, operator or clinician.

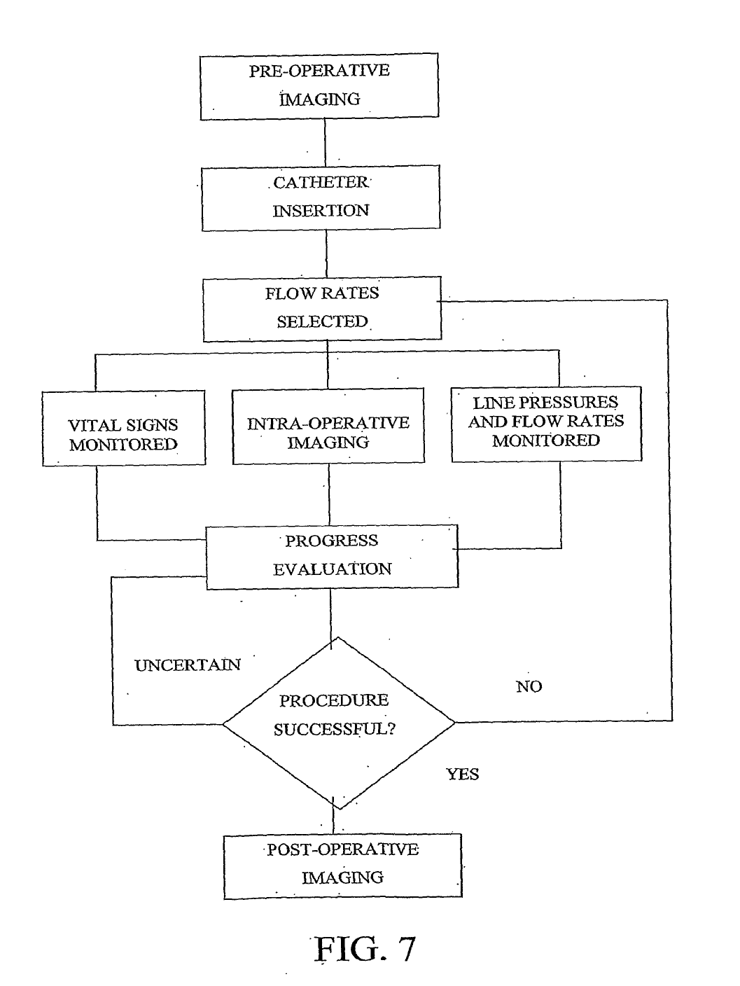

With regards to FIG. 7, FIG. 7 provides one possible version of the general class of algorithms that could be employed to control the overall system via the computer, processor or computational platform. The exemplary schema provides a flowchart of how a therapeutic treatment might be carried out using this system and related method.

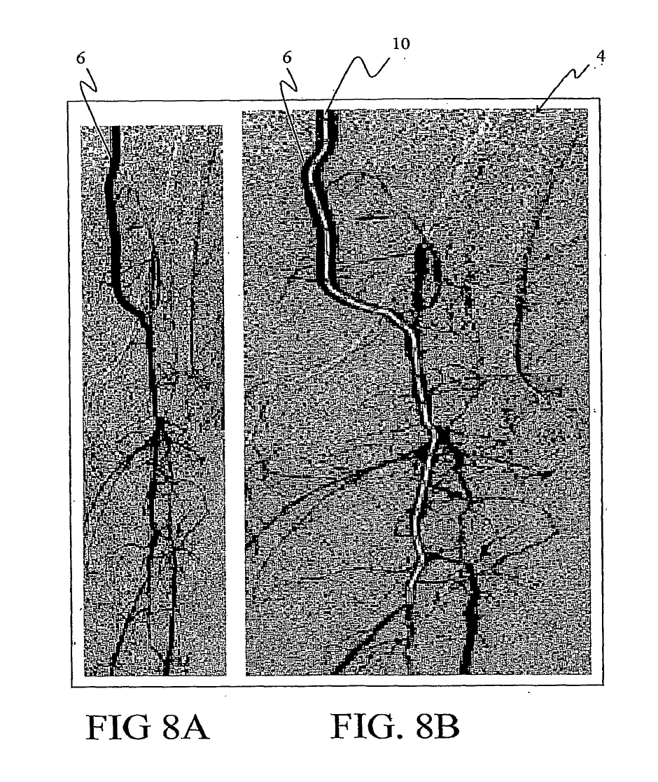

Referring generally to FIG. 8(A), FIG. 8(A) is a photographic depiction of a section of a peripheral vascular tract in a patient, into which an anti-thrombolytic agent may be delivered. Referring generally to FIG. 8(B), FIG. 8(B) is a subsequent photographic depiction of the multi-segmented, multi-port PeriCATH device of FIG. 8(A) wherein the device has now been navigated into the section of peripheral vasculature for the delivery of the agent. FIG. 8 shows a rendition of the disposition of a PeriCATH device 2 within a diseased artery 6 in the leg of a human patient 4. For illustration purposes, the catheter provided is the outer tube/catheter 10, but it should be appreciated that the inner tube/catheter 30 may have been provided instead. Moreover, it should be appreciated that both the inner and outer catheters may be utilized as discussed throughout. The design principles underlying the PeriCATH device can be implemented with the systems and devices disclosed in the following commonly owned U.S. patents and U.S. applications: U.S. Pat. No. 6,599,274, issued Jul. 29, 2003; Divisional U.S. application Ser. No. 10/444,884, filed May 23, 2003; CIP U.S. application Ser. No. 11/105,166, filed Apr. 13, 2005, and PCT Application No. US2006/013621, filed Apr. 12, 2006, of which are hereby incorporated by reference herein in their entirety. In the particular embodiment shown here, the PeriCATH is a multi-segmental outer catheter 10 with three maximum diameters, one for use within the superficial femoral artery, one for the popliteal, and one for the “run-off” vessels in the lower leg. These three segments gradually taper into each other. Their approximate diameters are 5.0 F, 3.5 F, and 2.0 F respectively. It should be appreciated that rather than distinct segments the catheter diameter may gradually taper. It should be appreciated that any catheter diameter size or length may be utilized as required or desired according to procedure or anatomy location.

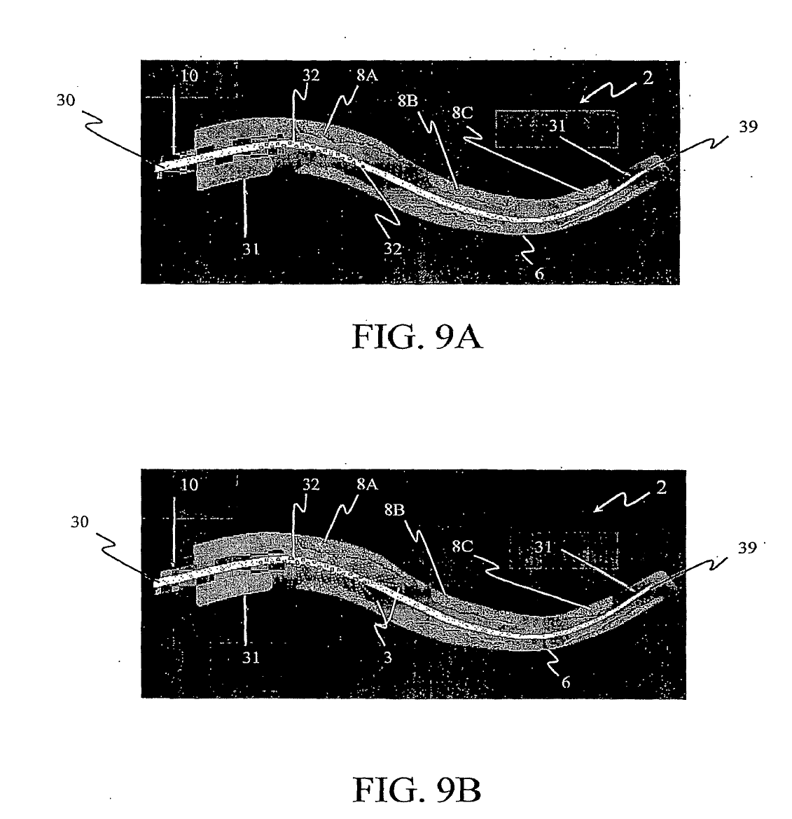

Generally referring to FIG. 9 (A)-(B), FIGS. 9 (A)-(B) are schematic depictions of a partial view of the tapered construction of the PeriCATH device or system with its position within an artery that has thrombal obstructions within it. FIGS. 9 (A)-(B) show the outer tube 10 having the inner tube 30 disposed therein with multiple ports 32 for fluid infusion. While the figure illustrates about twenty ports 32 it should be appreciated that any number greater or less than may be utilized as desired or required. In an embodiment, the inner tube 30 may be slightly smaller than 3.5 F. It should be appreciated that any catheter diameter size or length may be utilized as required or desired according to procedure or anatomy location. An optional flow-control balloon 31 is illustrated that is utilized to regulate the blood flow or medium flowing in a blood vessel (or applicable anatomical region) that circumferentially surrounds or proximal thereto the catheters. The flow-control balloon 31 may partially surround the inner and/or outer catheters and/or completely surround the outer or inner catheters. The balloon may also be any type of expandable compartment or structure.

The potential of infusing through different ports 32 may be applied for the proximal and middle segments of the inner catheter 30 (as well as outer catheter when applicable/desirable), or other segments as desired or required. Further, for example, the distal segment for infusing into the “run-off” vessels may be either used or not depending on the nature and location of the vascular occlusions 8a, 8b, and 8c. The baseline designs can include a single end-hole 39 in the inner tube 30 to infuse into this segment of the blood vessel 6. In an embodiment, multiple inner cores or lumens 30 will be available, each one having a different number and spacing of its ports 32. This will allow the operator to pick how many ports of the upper segment (or any applicable segments) and how many of the middle segment(s) (or any applicable segments) to infuse. Since the popliteal is a short artery, a small number of variations of the baseline design (in terms of port hole numbers and locations) may be all that is needed. Likewise for the femoral artery segment (although that is a longer segment). It should be appreciated that these design variations may dictate the number of components that would make up a complete assembly of PeriCATH elements for selection and use by the clinician in response to the anatomical and physiological requirements of the patient.

The medium can be transferred to the plurality of locations along the blood vessel 6. The transfer can be made at a single location 8a, 8b, or 8c, or a plurality of locations such as any two or more of the locations 8a, 8b, or 8c, thereby defining an elongated section. It should be appreciated that more than three locations may be effected as well. An elongated section may also be defined as a single occlusion, 8a, 8b, 8c if such is the case. In the case of a blood vessel, an elongated section may have, for example but not limited thereto, the following ranges in size: about 0.11 mm to 1 cm, about 1 cm to 10 cm, and over 10 cm. In the case of other body parts such as tubular structure or organ, an elongated section may have, for example but not limited thereto, the following ranges in size: about 0.1 mm to 1 cm, about 1 cm to 10 cm, and over 10 cm. It should appreciated that the catheter system may work on sections, locations, areas, or regions that are smaller/less than 0.1 mm or much larger/greater than 10 cm according to the anatomical and physiological requirements of the patient.

It should be appreciated that the system may be implemented within a plurality of locations in the body. The body part may be an organ or tubular structure. An organ includes, for example, a hollow organ, solid organ, parenchymal tissue and/or stromal tissue. Such hollow organs may be, for example but not limited thereto, 5-stomach, esophagus, colon, rectum, and ducts, or the like. A tubular structure may include a blood vessel. A blood vessel may include one or more of the following: vein, venule, artery, arterial, or capillary.

In another preferred embodiment, the PeriCATH may be a non-segmental multi-port perfusion catheter for carrying out infusions into the organ, wherein the organ may include solid organs like the liver (hepatomas), pancreas, etc. The design would insure the capability of creating other uniform or variable distributions into the solid organ, depending on the flow paths chosen through the various multiple ports.

In still another preferred embodiment, the PeriCATH would be an endovascular catheter with a plurality of microcoils on the distal tip or distributed along it axially, for enhancement of the quality of MR imaging. This version of the device would be used either with or without a plurality of surface coils. In an approach, the endovascular coil may not be segmented, but rather a straight-catheter with multiple ports. This embodiment of the device could also be used in solid or hollow organs, in addition to endovascular imaging of intima and vessel walls.

It should be appreciated that imaging system may be a variety of applicable imaging systems, such as but not limited thereto the following: magnetic resonance imaging (MRI), magnetic resonance angiography, functional magnetic resonance imaging, interventional magnetic resonance imaging, biplanar fluoroscopy, CT, nuclear medicine cameras, standard x-ray imaging, Position Emission Tomography (PET scans), and/or other such imaging systems.

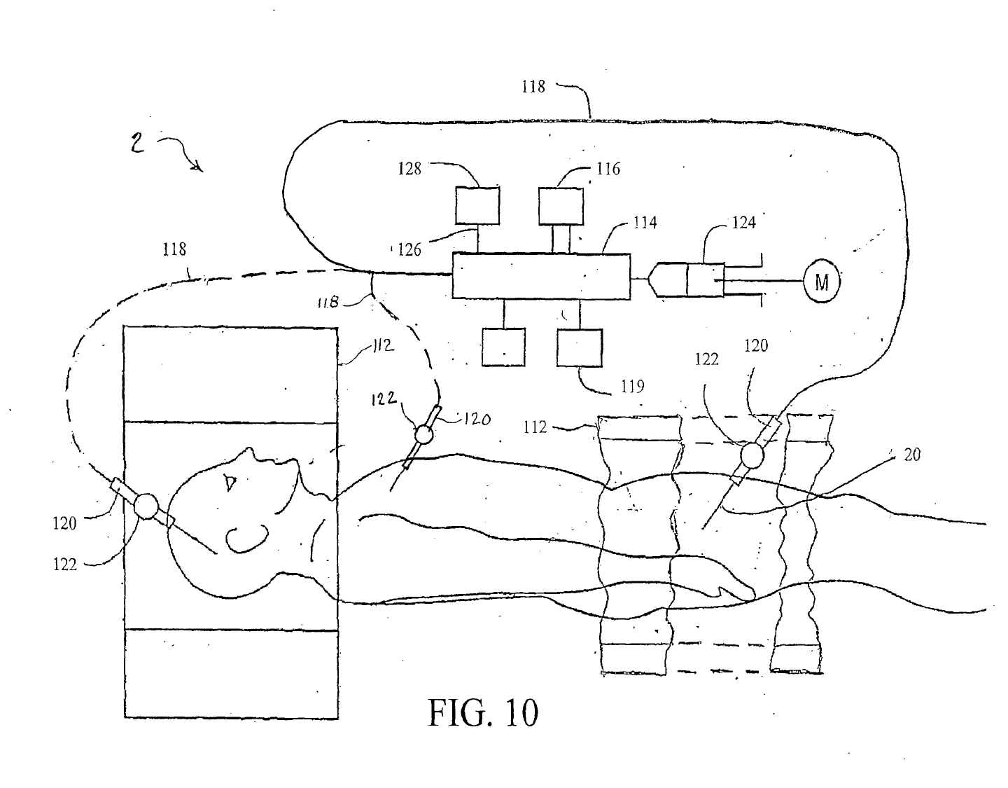

FIG. 10 is a schematic diagram showing a patient 4, or any subject or object, undergoing an examination and/or intervention inside the bore of an MRI system 112 whereby a catheter device is disposed at any of one or more desired or required locations within the patient. In an approach, a manifold 114 couples several therapeutic or diagnostic devices typified by device 116 to the catheter system 2. A syringe, flow-driver or pumping device 124 is also in communication with the manifold 114. The catheter system 2 in turn may be delivered through a guide sheath 120 that may be positioned in a navigation guide 122. In operation the physician or user inserts one or more such catheter system 2 into body, for instance on going into the leg, chest or skull (or other anatomical part or parts or subject region or regions to cover the hollow or solid organs, blood vessels, etc.) under MRI guidance or other applicable examination or intervention. The same or similar MRI visualization may be used to follow the progress of the one or more implant(s) both acutely and chronically. This catheter device may have various interior and peripheral lumens, chambers and channels. Such interior and peripheral lumens, chambers and channels may be used to deliver other devices and perform various diagnostic functions. For example, each lumen, chamber, and channel may communicate with a separate port of the manifold 114. A lumen, chamber or channel may contain a pressure transducer 128. Other lumens and channels may be devoted to an optical or other type of cell counter device, for example, as shown generically as device 119 in FIG. 10. Such a device may operate with two fibers located in two separate lumens and/or ports to measure the number of and viability of cells delivered by the catheter. An example of fiber optics related application/technology is discussed in U.S. patent application Ser. No. 10/444,884, filed May 23, 2003 (U.S. Application No. 2003/0204171, published Oct. 30, 2003), and of which are hereby incorporated by reference herein in their entirety.

It should be appreciated that many other embodiments of inner and outer tube means, port hole means, sealing rings, sealing plates and baffle means, endcap means, taper and distal port hole means, flow channeling and recirculation means, microcoil means, pump means, pressure and flow-rate monitor means, imaging means, computer means, and other details of construction and use constitute non-inventive variations of the novel and insightful conceptual means, system, and technique which underlie the present invention. An example of systems and methods that may be implemented with various embodiments of the present invention are provided in the following commonly owned Applications: U.S. patent application Ser. No. 10/444,884, filed May 23, 2003 (US Application No. 2003/0204171, published Oct. 30, 2003); PCT Application No. PCT/US2005/026738, filed Jul. 28, 2005; and PCT Application No. 2006/005876, filed Feb. 16, 2006, and of which are hereby incorporated by reference herein in their entirety.

It should be appreciated that as discussed herein; a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g.; a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

We claim:

1. A catheter system for transferring at least one medium into a plurality of locations within a body of a patient, said system comprising:
an outer tube having a distal end and a proximal end, and having a wall extending longitudinally there between;
at least one inner tube disposed within said outer tube, said at least one inner tube having a distal end and a proximal end, and having a wall extending longitudinally there between;
said at least one inner tube being adapted to move longitudinally and/or circumferentially relative to said outer tube to move along the body to allow transference of the medium between said inner tube and said plurality of locations within the body;
an imaging system to provide imaging data; and
a medium control system to control the medium.

2. The system of claim 1, said medium control system comprising:
a regulator system to regulate the pressures and/or flows of medium to the plurality of locations.

3. The system of claim 2, said medium control system further comprising:
a computer processor and a feedback system.

4. The system of claim 1 wherein said transfer of the medium to the plurality of locations within the body is accomplished simultaneously.

5. The system of claim 1 wherein said transfer of the medium is accomplished in a scheduled combination of transfers that is clinically desirable.

6. The system of claim 1 wherein said medium control system is a regulator system and/or a feedback system.

7. The system of claim 1, further comprising;
at least one outer tube aperture disposed on said outer tube wall; and
at least one inner tube aperture disposed on said inner tube wall.

8. The system of claim 7, wherein said at least one inner tube being adapted to move longitudinally and/or circumferentially relative to said outer tube to provide for said at least one inner tube aperture to be in communication with said at least one outer tube aperture.

9. The system of claim 7, wherein said at least one inner tube aperture and/or said at least one outer tube aperture may be a recess, port, duct, trough, bore, inlet, hole, perforation, channel, passage, slot, orifice, porthole, conduit, or semi-permeable structure.

10. The system of claim 1, wherein at least a portion of said outer tube wall is a semipermeable membrane.

11. The system of claim 1, wherein at least a portion of said inner tube wall is a semipermeable membrane.

12. The system of claim 1, wherein said medium control system comprises:
at least one pump device,
at least one pressure monitoring device; and
at least one flow-rate measurement device, said flow-rate measurement device adapted to measure amounts of the medium traveling in said one or more inner tubes.

13. The system of claim 1, wherein said patient comprises a human or any animal.

14. The system of claim 1, wherein said outer tube comprises a variable diameter along its axial length.

15. The system of claim 14, wherein said variable diameter outer tube comprises a multi-segment tube.

16. The system of claim 14, wherein said variable diameter outer tube has a diameter that increases and/or decreases.

17. The system of claim 1, wherein said outer tube comprises a fixed diameter.

18. The system of claim 1, wherein said inner tube comprises a variable diameter along its axial length.

19. The system of claim 18, wherein said variable diameter inner tube comprises a multi-segment tube.

20. The system of claim 18, wherein said variable diameter inner tube has a diameter that increases and/or decreases.

21. The system of claim 1, wherein said inner tube comprises a fixed diameter.

22. The system of claim 1, wherein two or more of said at least one inner tube comprise non-communicating channels for the transfer of one or more mediums to the plurality of locations.

23. The system of claim 1, wherein the transference of one or more mediums to the plurality of locations is effected by said medium control system that independently selects pressures and/or flow rates under the guidance of the clinician operating it.

24. The system of claim 1, wherein the transferring of the medium comprises delivering the medium from said inner tube to the body.

25. The system of claim 24, wherein said delivered medium comprises at least one of thrombolytic agents, chemotherapies, cell slurries, gene therapy vectors, growth factors, angiogenesis factors, radionuclide slurries, anti-infection agents, anti-tumor compounds, apoptosis-inducing agents, receptor-bound agents, brachytherapeutic agents, antiproliferative agents, cleansing or ravaging agents, and/or other types of drugs, therapies, or agents.

26. The system of claim 24, wherein said delivered medium comprises at least one of contrast agents, diagnostic agents, cleansing or ravaging agents, and/or therapeutic agents.

27. The system of claim 1, wherein the body comprises an organ.

28. The system of claim 27, wherein said organ comprises hollow organs, parenchymal tissue, stromal tissue, and/or ducts.

29. The system of claim 1, wherein said body comprises a tubular structure.

30. The system of claim 29, wherein said tubular structure comprises a blood vessel.

31. The system of claim 1, wherein the transferring of the medium comprises withdrawing the medium from the body to said inner tube.

32. The system of claim 31, wherein said withdrawn medium comprises at least one of edematous fluids, blood, cerebrospinal fluid, interstitial fluid, infected materials, neoplastic fluids and tissues, thrombolysis byproducts including clot fragments and the like, metabolic byproducts, excess drugs and agents, and other such substances.

33. The system of claim 1, wherein the longitudinal and/or circumferential movement of the inner tube determines the location of transfer of the medium to the plurality of locations.

34. The system of claim 1, wherein the longitudinal and/or circumferential movement of the inner tube determines the aperture size and therefore, at least in part, the transfer rate.

35. The system of claim 1, wherein said inner tube further comprises an endport at said distal end of said inner tube, wherein said end port is adapted for said medium to transfer through to the plurality of locations.

36. The system of claim 1, wherein said at least one inner tube at least partially extends out from the distal end of said outer tube during the transferring.

37. The system of claim 1, wherein said at least one inner tube does not extend out from the distal end of said outer tube during transferring.

38. The system of claim 1, further comprising at least one microcoil system in communication with said catheter system for imaging.

39. The system of claim 38, wherein said at least one microcoil system is disposed on said inner tube and/or said outer tube.

40. The system of claim 1, wherein said imaging system comprises at least one of: magnetic resonance imaging, magnetic resonance angiography, functional magnetic resonance imaging (MRI), interventional magnetic resonance imaging, biplanar fluoroscopy, CT, nuclear medicine cameras, standard x-ray imaging, Position Emission Tomography (PET scans), and other such imaging systems.

41. The system of claim 1, wherein the plurality of locations comprises: a thrombotic lesion or other type of occlusive lesion or plurality of such lesions.

42. The system of claim 1, wherein said medium control system provides flow rate and pressure data from said one or more inner tubes.

43. The system of claim 1, further comprising imaging contrast enhancement agents that are provided at the plurality of target locations.

44. The system of claim 1, wherein the plurality of locations are located at an elongated section of the body, wherein said outer tube remains at least substantially fixed in position during the transferring of the at least one medium to the elongated section.

45. The system of claim 44, wherein said body comprises a blood vessel and said elongated section of the blood vessel is at least about 0.1 mm to about 1 cm.

46. The system of claim 44, wherein said body comprises a blood vessel and said elongated section of the blood vessel is at least about 1 cm to about 10 cm.

47. The system of claim 44, wherein said body comprises a blood vessel and said elongated section of the blood vessel is greater than about 10 cm.

48. The system of claim 44, wherein said body comprises a tubular structure and said elongated section of the tubular structure is at least about 0.1 mm to about 1 cm.

49. The system of claim 44, wherein said body comprises a tubular structure and said elongated section of the tubular structure is at least about 1 cm to about 10 cm.

50. The system of claim 44, wherein said body comprises a tubular structure and said elongated section of the tubular structure is greater than about 10 cm.

51. The system of claim 44, wherein said body comprises an organ and said elongated section of the organ is at least about 0.1 mm to about 1 cm.

52. The system of claim 44, wherein said body comprises an organ and said elongated section of the organ is at least about 1 cm to about 10 cm.

53. The system of claim 44, wherein said body comprises an organ and said elongated section of the organ is at greater than about 10 cm.

54. The system of claim 1 further comprising a guidewire.

55. The system of claim 54, wherein said guidewire is used for guiding said at least one inner tube.

56. The system of claim 1, wherein the transfer of the medium comprises transferring a therapeutic agent and/or a diagnostic agent separately to two or more different said pluralities of locations in the body.

57. The system of claim 1, further comprising at least one flow-control compartment that at least partially surrounds said inner tube and/or said outer tube.

58. A method for transferring at least one medium into a plurality of locations within a body of a patient using a catheter system, said method comprising:
inserting an outer tube into the body, said outer tube having a distal end and a proximal end, and having a wall extending longitudinally there between;
inserting at least one inner tube disposed within said outer tube, said at least one inner tube having a distal end and a proximal end, and having a wall extending longitudinally there between;
said at least one inner tube being adapted to move longitudinally and/or circumferentially relative to said outer tube to move along the body to allow transference of the medium between said inner tube and said plurality of locations within the body;
imaging data using an imaging system; and
controlling the medium.

59. The method of claim 58, said controlling of the medium comprises:
regulating the pressures and/or flows of medium to the plurality of locations.

60. The method of claim 59, said medium of controlling the medium further comprising:
providing a computer processor and a feedback system.

61. The method of claim 58 wherein said transferring of the medium to the plurality of locations within the body is accomplished simultaneously.

62. The method of claim 58 wherein said transferring of the medium is accomplished in a scheduled combination of transfers that is clinically desirable.

63. The M of claim 58 wherein said controlling the medium is accomplished using a medium control system, wherein said medium control system is a regulator system and/or a feedback system.

64. The method of claim 58, further comprising:
at least one outer tube aperture disposed on said outer tube wall; and
at least one inner tube aperture disposed on said inner tube wall.

65. The method of claim 64, wherein said at least one inner tube being adapted to move longitudinally and/or circumferentially relative to said outer tube to provide for said at least one inner tube aperture to be in communication with said at least one outer tube aperture.

66. The method of claim 64, wherein said at least one inner tube aperture and/or said at least one outer tube aperture may be a recess, port, duct, trough, bore, inlet, hole, perforation, channel, passage, slot, orifice, porthole, conduit, or semi-permeable structure.

67. The method of claim 58, wherein at least a portion of said outer tube wall is a semipermeable membrane.

68. The method of claim 58, wherein at least a portion of said inner tube wall is a semipermeable membrane.

69. The method of claim 58, wherein said controlling the medium is accomplished with a medium control system, said medium control system comprises:
at least one pump device,
at least one pressure monitoring device; and
at least one flow-rate measurement device, said flow-rate measurement device adapted to measure amounts of the medium traveling in said one or more inner tubes.

70. The method of claim 58, wherein said patient comprises a human or any animal.

71. The method of claim 58, wherein said outer tube comprises a variable diameter along its axial length.

72. The method of claim 71, wherein said variable diameter outer tube comprises a multi-segment tube.

73. The method of claim 71, wherein said variable diameter outer tube has a diameter that increases and/or decreases.

74. The method of claim 58, wherein said outer tube comprises a fixed diameter.

75. The method of claim 58, wherein said inner tube comprises a variable diameter along its axial length.

76. The method of claim 75, wherein said variable diameter inner tube comprises a multi-segment tube.

77. The method of claim 75, wherein said variable diameter inner tube has a diameter that increases and/or decreases.

78. The method of claim 58, wherein said inner tube comprises a fixed diameter.

79. The method of claim 58, wherein two or more of said at least one inner tube comprise non-communicating channels for the transferring of one or more mediums to the plurality of locations.

80. The method of claim 58, wherein the transferring of one or more mediums to the plurality of locations is effected by said controlling the medium comprises: independently selecting pressures and/or flow rates under the guidance of the clinician operating it.

81. The method of claim 58, wherein the transferring of the medium comprises delivering the medium from said inner tube to the body.

82. The method of claim 81, wherein said delivered medium comprises at least one of thrombolytic agents, chemotherapies, cell slurries, gene therapy vectors, growth factors, angiogenesis factors, radionuclide slurries, anti-infection agents, anti-tumor compounds, apoptosis-inducing agents, receptor-bound agents, brachytherapeutic agents, antiproliferative agents, cleansing or ravaging agents, and/or other types of drugs, therapies, or agents.

83. The method of claim 81, wherein said delivered medium comprises at least one of contrast agents, diagnostic agents, cleansing or lavaging agents, and/or therapeutic agents.

84. The method of claim 58, wherein the body comprises an organ.

85. The method of claim 84, wherein said organ comprises hollow organs, parenchymal tissue, stromal tissue, and/or ducts.

86. The method of claim 58, wherein said body comprises a tubular structure.

87. The method of claim 86, wherein said tubular structure comprises a blood vessel.

88. The method of claim 58, wherein the transferring of the medium comprises withdrawing the medium from the body to said inner tube.

89. The method of claim 88, wherein said withdrawn medium comprises at least one of edematous fluids, blood, cerebrospinal fluid, interstitial fluid, infected materials, neoplastic fluids and tissues, thrombolysis byproducts including clot fragments and the like, metabolic byproducts, excess drugs and agents, and other such substances.

90. The method of claim 58, wherein the longitudinal and/or circumferential movement of the inner tube determines the location of transfer of the medium to the plurality of locations.

91. The method of claim 58, wherein the longitudinal and/or circumferential movement of the inner tube determines the aperture size and therefore, at least in part, the transfer rate.

92. The method of claim 58, wherein said inner tube further comprises an endport at said distal end of said inner tube, wherein said end port is adapted for said medium to transfer through to the plurality of locations.

93. The method of claim 58, wherein said at least one inner tube at least partially extends out from the distal end of said outer tube during the transferring.

94. The method of claim 58, wherein said at least one inner tube does not extend out from the distal end of said outer tube during transferring.

95. The method of claim 58, further comprising providing at least one microcoil system in communication with said catheter system for imaging.

96. The method of claim 95, wherein said at least one microcoil system is disposed on said inner tube and/or said outer tube.

97. The method of claim 58, wherein said imaging comprises an imaging system comprising at least one of: magnetic resonance imaging, magnetic resonance angiography, functional magnetic resonance imaging (MRI), interventional magnetic resonance imaging, biplanar fluoroscopy, CT, nuclear medicine cameras, standard x-ray imaging, Position Emission Tomography (PET scans), and other such imaging systems.

98. The method of claim 58, wherein the plurality of locations comprises: a thrombotic lesion or other type of occlusive lesion or plurality of such lesions.

99. The method of claim 58, wherein said controlling the medium is accomplished with a medium control system, said medium control system provides flow rate and pressure data from said one or more inner tubes.

100. The method of claim 58, further comprising providing imaging contrast enhancement agents that are provided at the plurality of target locations.

101. The method of claim 58, wherein the plurality of locations are located at an elongated section of the body, wherein said outer tube remains at least substantially fixed in position during the transferring of the at least one medium to the elongated section.

102. The method of claim 10, wherein said body comprises a blood vessel and said elongated section of the blood vessel is at least about 0.1 mm to about 1 cm.

103. The method of claim 101, wherein said body comprises a blood vessel and said elongated section of the blood vessel is at least about 1 cm to about 10 cm.

104. The method of claim 101, wherein said body comprises a blood vessel and said elongated section of the blood vessel is greater than about 10 cm.

105. The method of claim 10, wherein said body comprises a tubular structure and said elongated section of the tubular structure is at least about 0.1 mm to about 1 cm.

106. The method of claim 101, wherein said body comprises a tubular structure and said elongated section of the tubular structure is at least about 1 cm to about 10 cm.

107. The method of claim 101, wherein said body comprises a tubular structure and said elongated section of the tubular structure is greater than about 10 cm.

108. The method of claim 101, wherein said body comprises an organ and said elongated section of the organ is at least about 0.1 mm to about 1 cm.

109. The method of claim 101, wherein said body comprises an organ and said elongated section of the organ is at least about 1 cm to about 10 cm.

110. The method of claim 101, wherein said body comprises an organ and said elongated section of the organ is at greater than about 10 cm.

111. The method of claim 58, further comprising:
guiding said inner tube and/or outer tube using one or more guidewires.

112. The method of claim 111, wherein said guidewire is used for guiding said at least one inner tube.

113. The method of claim 58, wherein the transferring of the medium comprises transferring a therapeutic agent and/or a diagnostic agent separately to two or more different said pluralities of locations in the body.

114. The method of claim 58, further comprising controlling the flow of blood, fluid or material outside said inner tube and/or said outer tube.
